(19) United States
(12) Patent Application Publication
(10) Pub. No.: US 2024/0384213 A1
(43) Pub. Date: Nov. 21, 2024

(54) BIOREACTOR FOR SUSPENSION CELL

(71) Applicant: AMOGREENTECH CO., LTD., Gimpo-si (KR)

(72) Inventors: Seon Ho JANG, Gimpo-si (KR); Kyoung Ku HAN, Gimpo-si (KR); In Yong SEO, Gimpo-si (KR); Seoung Hoon LEE, Gimpo-si (KR); Jae Kyung SONG, Gimpo-si (KR); Song Hee KOO, Gimpo-si (KR); Hee Sung PARK, Gimpo-si (KR); Hyeong Tak NOH, Gimpo-si (KR); Ji Young KIM, Gimpo-si (KR); Su Yeon LEE, Gimpo-si (KR); Seung Min CHO, Gimpo-si (KR)

(73) Assignee: AMOGREENTECH CO., LTD., Gimpo-si (KR)

(21) Appl. No.: 18/692,853

(22) PCT Filed: Sep. 15, 2022
(86) PCT No.: PCT/KR2022/013820

§ 371 Date: Mar. 18, 2024

(30) Foreign Application Priority Data
Sep. 16, 2021 (KR) 10-2021-0124295

(51) Int. Cl. C12M 1/12; C12M 1/00

(52) U.S. Cl. CPC C12M 25/02; C12M 23/04; C12M 23/34; C12M 25/14; C12M 25/16

(57) ABSTRACT

A bioreactor for suspension cells is provided. The bioreactor for suspension cells may comprise: a frame unit; a driving unit mounted on the frame unit, and a cell culture unit including a housing having an inner space filled with a medium containing suspension cells, and a plurality of supports disposed in multiple stages in the inner space to be spaced at intervals from each other, the cell culture unit having one end axially coupled to the driving unit.

### CROSS REFERENCE TO THE RELATED APPLICATIONS

This application is the national phase entry of International Application No. PCT/KR2022/013820, filed on Sep. 15, 2022, which is based upon and claims priority to Korean Patent Application No. 10-2021-0124295, filed on Sep. 16, 2021, the entire contents of which are incorporated herein by reference.

### TECHNICAL FIELD

The present invention relates to a bioreactor for suspension cells.

### BACKGROUND

A bioprocess is a process in which living cells are used to produce a desired therapeutic agent in the field of biology.

Antibodies, stem cells, immune cells, and the like are produced through cell culture and are used to produce biomedicine, vaccines, and cell therapy products.

Cells are classified according to their adhesive abilities into adherent cells that should be attached to a substrate and suspension cells that can proliferate without being attached to the substrate.

That is, while adherent cells are cultured while attached to a scaffold which serves as a substrate, suspension cells are not attached to the scaffold when cultured and grow by receiving signals through repeating a process of contact-suspension-contact-suspension in which the suspension cells are attached to a surface of the scaffold, detached therefrom, attached to the surface of the scaffold again, and detached therefrom.

Such suspension cells are very sensitive to stress and thus are cultured using a medium that does not move within a predetermined culture space.

However, the above-described repeated contact of suspension cells to a scaffold is required for smooth growth of the suspension cells, but since the medium does not move within the culture space, there is a problem in that the suspension cells cannot contact easily the scaffold.

### SUMMARY

The present invention is directed to providing a bioreactor for suspension cells that can promote movement of suspension cells suspended in a medium.

The present invention is also directed to providing a bioreactor for suspension cells that can culture a large amount of suspension cells even at a small size.

The present invention provides a bioreactor for suspension cells, the bioreactor including: a frame part; a driving part mounted on the frame part; and a cell culture part including a housing having an inner space filled with a medium including suspension cells and a plurality of scaffolds disposed in multiple stages and spaced predetermined intervals from each other in the inner space, the cell culture part having one side axially coupled to the driving part.

Also, the cell culture part may swing through driving of the driving part for the medium to be moved in a space between two scaffolds neighboring each other.

Also, the cell culture part may further include a gas inlet formed to pass through the housing and having a predetermined area to allow a gas to be introduced from the outside into the inner space and a porous member configured to cover the gas inlet to allow the gas to be introduced from the outside into the inner space while preventing the medium filled in the inner space from leaking to the outside. In this case, the porous member may be a water-repellent membrane.

Also, the scaffold may be a plate-shaped member having a predetermined area, the inner space may be divided into a plurality of culture spaces through the plurality of scaffolds disposed in multiple stages and spaced predetermined intervals in one direction of the housing, and the medium including the suspension cells may be stored in the inner space to fill each of the plurality of culture spaces.

Also, the scaffold may include a plate-shaped support member having a predetermined area and a pair of nanofiber membranes attached to both surfaces of the support member via an adhesive layer, and the nanofiber membranes may be plate-shaped nanofiber membranes that are motif-coated.

Also, the scaffold may further include a plurality of through-holes formed to pass through the support member to facilitate passage of the gas introduced from the outside into the inner space.

Also, the plurality of scaffolds may remain spaced predetermined intervals via a spacing member disposed between two scaffolds.

In addition, the medium may further include magnetic particles that are peptide motif-coated.

According to the present invention, movement of suspension cells suspended in a medium is promoted to cause more cell-to-cell interactions and peptide signals. Accordingly, the growth of the suspension cells can be promoted.

### BRIEF DESCRIPTION OF THE DRAWINGS

FIG. 1 is a schematic diagram illustrating a bioreactor for suspension cells according to one embodiment of the present invention.

FIG. 2 is a view schematically illustrating the connection relation between a driving part and a cell culture part in the bioreactor for suspension cells according to one embodiment of the present invention.

FIG. 3 is an operational state view of FIG. 1.

FIG. 4 is an exploded view of major components of the cell culture part that may be applied to FIG. 1.

FIG. 5 is an exploded view of a scaffold assembly illustrated in FIG. 4.

FIG. 6 is a cross-sectional view in direction A-A of FIG. 1.

FIG. 7 is a view illustrating one form of a scaffold that may be used in the bioreactor for suspension cells according to one embodiment of the present invention.

FIG. 8 is a view illustrating another form of a scaffold that may be used in the bioreactor for suspension cells according to one embodiment of the present invention.

### DETAILED DESCRIPTION OF THE EMBODIMENTS

Hereinafter, embodiments of the present invention will be described in detail with reference to the accompanying drawings so that those of ordinary skill in the art to which the present invention pertains can easily carry out the present invention. The present invention may be implemented in various different forms and is not limited to the embodiments described herein. In the drawings, in order to clearly describe the present invention, parts irrelevant to the description are omitted, and like or similar components are denoted by like reference numerals throughout the specification.

A bioreactor 100 for suspension cells according to one embodiment of the present invention may include a plurality of scaffolds 136 and 136′ for cell culture, and the plurality of scaffolds 136 and 136′ may be disposed in multiple stages and spaced predetermined intervals from each other in an inner space S.

Also, in the bioreactor 100 for suspension cells according to one embodiment of the present invention, a medium supplied from the outside may be filled in a culture space S1 formed between the two scaffolds 136 and 136′ spaced predetermined intervals from each other in the inner space S and may also be filled in the inner space S for all of the plurality of scaffolds 136 and 136′ to be submerged.

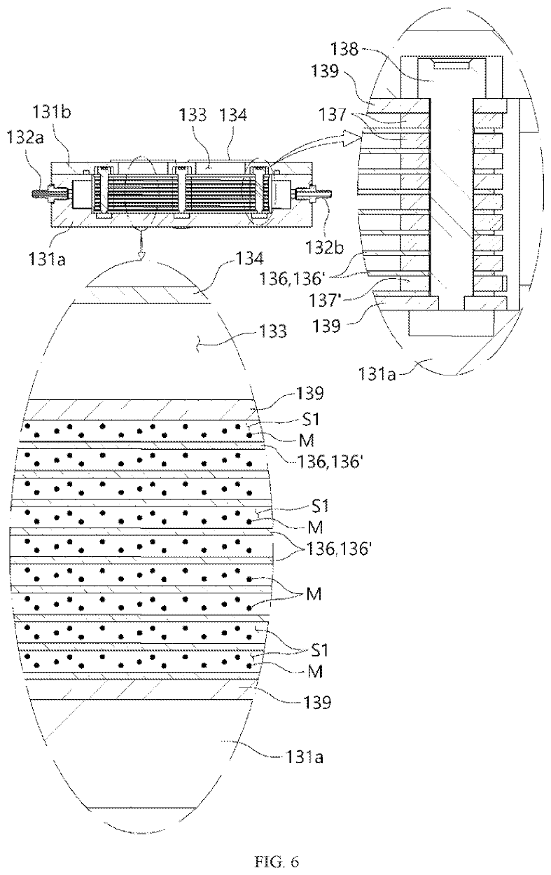

Here, the medium may be a medium including suspension cells to be cultured, and as illustrated in FIG. 6, the medium may further include, in addition to the suspension cells, magnetic particles that are peptide motif-coated.

Accordingly, the suspension cells included in the medium may repeat a process of being in contact with the scaffolds 136 and 136′ and then detached therefrom while suspended in the culture space S1 formed between the two scaffolds 136 and 136′, and by repeating the process of being in contact with and then detached from the scaffolds 136 and 136′, the suspension cells may receive signals from the scaffolds 136 and 136′ and grow.

In this case, the bioreactor 100 for suspension cells according to one embodiment of the present invention can promote movement of the suspension cells by causing minimum movement of the medium filled in the culture space S1.

Accordingly, the bioreactor 100 for suspension cells according to one embodiment of the present invention can promote growth of suspension cells by allowing more cell-to-cell interactions and peptide signals while not interfering with cell growth caused by movement of the medium.

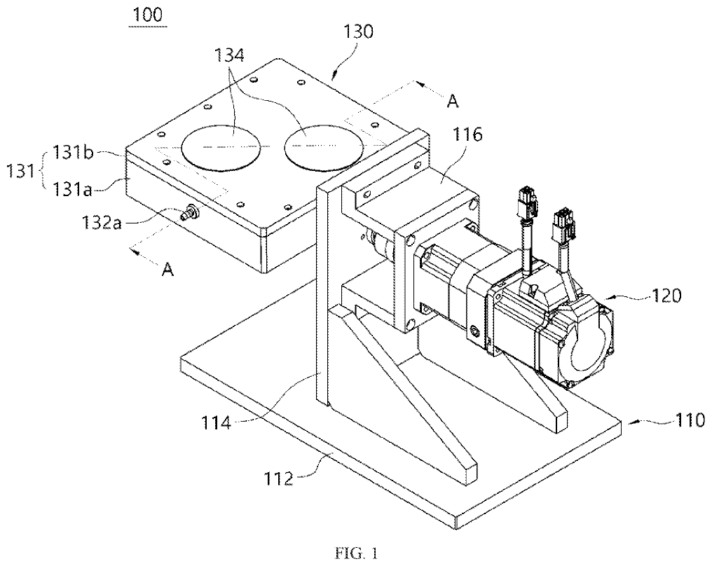

To this end, as illustrated in FIGS. 1 to 3, the bioreactor 100 for suspension cells according to one embodiment of the present invention includes a frame part 110, a driving part 120, and a cell culture part 130.

The driving part 120 may be mounted on the frame part 110, and the cell culture part 130 may have one side axially coupled to the driving part 120.

That is, in the bioreactor 100 for suspension cells according to one embodiment of the present invention, since the driving part 120 is axially coupled to the one side of the cell culture part 130 while fixed to the frame part 110, the cell culture part 130 may reciprocate and swing through driving of the driving part 120.

Accordingly, the medium filled in the cell culture part 130 may be moved in the cell culture part 130 through a reciprocating swinging motion of the cell culture part 130, and the suspension cells included in the medium may easily come into contact with the scaffolds 136 and 136′ by movement of the suspension cells being promoted by the movement of the medium.

To this end, as illustrated in FIGS. 1 to 3, the frame part 110 may include a plate-shaped base plate 112 having a predetermined area and a mounting plate 114 extending a predetermined height from the base plate 112.

In such a case, the driving part 120 may be fixed to the mounting plate 114 via a fixing bar 116 fixed to the mounting plate 114.

Here, the driving part 120 may be a known driving motor in which a rotating shaft 122 reciprocates in both directions when power is applied, and the driving part 120 may include a reducer connected to the driving motor to control a rotational speed of the rotating shaft 122.

In this case, the cell culture part 130 may be axially coupled to the rotating shaft 122 of the driving part 120 via a power transmission member 135 provided at the one side of the cell culture part 130.

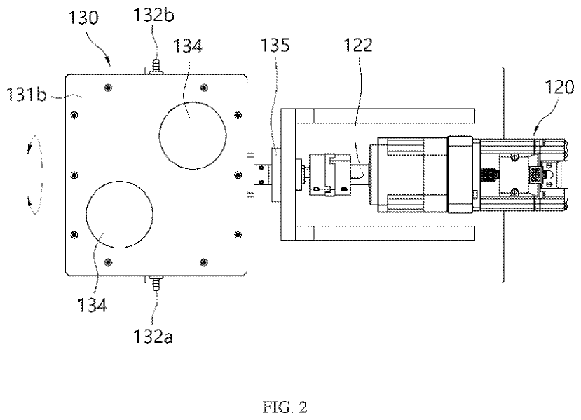

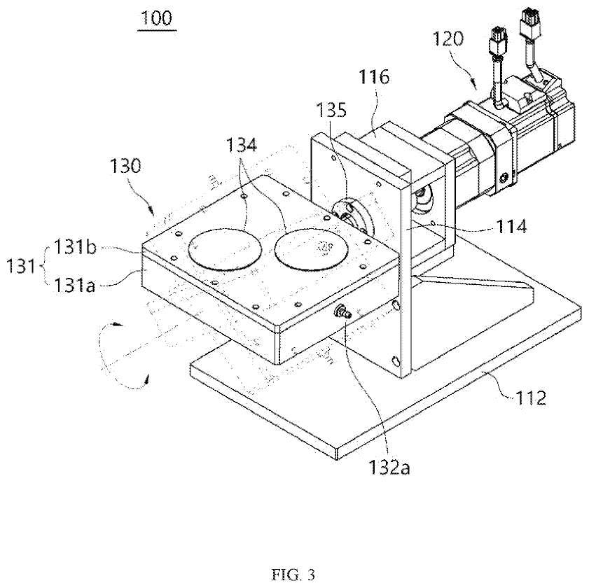

Accordingly, the driving part 120 and the cell culture part 130 axially coupled to each other may be installed on the mounting plate 114 while spaced a predetermined height from the base plate 112 of the frame part 110, and as illustrated in FIGS. 2 and 3, the cell culture part 130 may easily swing in both directions by receiving a driving force through the power transmission member 135 when the rotating shaft 122 rotates.

As a result, as described above, the medium including the suspension cells that is filled in the cell culture part 130 may be moved by swinging of the cell culture part 130, and the suspension cells included in the medium may be easily in contact with the scaffolds 136 and 136′ by movement of the suspension cells being promoted by the movement of the medium.

As described above, the cell culture part 130 may have one side axially coupled to the driving part 120 via the power transmission member 135 and include the plurality of scaffolds 136 and 136′ for cell culture, and the plurality of scaffolds 136 and 136′ may be disposed in multiple stages inside the cell culture part 130.

To this end, the cell culture part 130 may include a housing 131 and the plurality of scaffolds 136 and 136′.

The housing 131 may accommodate therein the plurality of scaffolds 136 and 136′ and the medium. To this end, the housing 131 may be formed in the shape of a box having the inner space S.

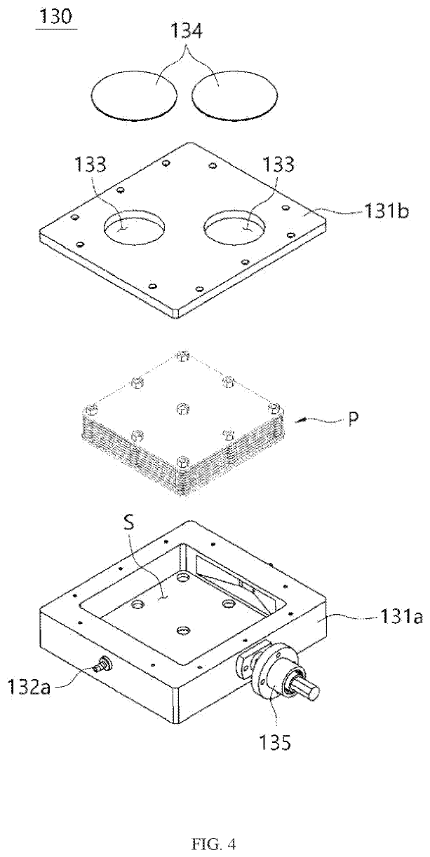

For example, as illustrated in FIG. 4, the housing 131 may include a box-shaped body 131a having the inner space S and an open upper surface and a cover 131b configured to cover the open upper portion of the body 131a.

In such a case, the housing 131 may include an inlet 132a provided at one side of the body 131a for the medium supplied from the outside to be introduced into the inner space S and an outlet 132b provided at one side of the body 131a for the medium of the inner space S to be discharged to the outside, and the power transmission member 135 axially coupled to the rotating shaft 122 of the driving part 120 may be provided at one side of the body 131a.

Accordingly, the inner space S may be filled with the medium supplied from the outside through the inlet 132a, the medium may be discharged to the outside through the outlet 132b after cell culture is completed, and the body 131a may swing through driving of the driving part 120.

Although the inlet 132a and the outlet 132b are illustrated in the drawings and described as being separately provided at both sides of the body 131a, the present invention is not limited thereto, and the inlet 132a and the outlet 132b may also be integrated into one port.

In this case, the bioreactor 100 for suspension cells according to one embodiment of the present invention may include at least one gas inlet 133 communicating with the inner space S for a gas to be introduced toward the medium filled in the inner space S.

For example, the gas inlet 133 may be formed to pass through the cover 131b of the housing 131.

Accordingly, during cell culture using the bioreactor 100 for suspension cells according to one embodiment of the present invention, when the bioreactor 100 for suspension cells is disposed inside a chamber such as an incubator, a gas present in the incubator may be supplied through the gas inlet 133 to the inner space S of the housing 131 filled with the medium.

Here, the incubator may be a space providing a culture environment of the suspension cells included in the medium. For example, the incubator may be a chamber, and the inside of the chamber may be an environment in which a temperature and a concentration of CO₂ are constantly maintained.

Also, the gas may be CO₂ gas but is not limited thereto and may be appropriately changed according to cells to be cultured.

In such a case, the incubator may include an air conditioning system for maintaining the internal temperature at a certain temperature and may further include a gas supply device (not illustrated) or the like configured to stably supply a gas necessary for cell culture into the incubator to maintain a concentration of the gas inside the incubator at a certain level.

Accordingly, in the bioreactor 100 for suspension cells that is disposed inside the incubator, the gas present in the incubator may be introduced into the inner space S of the housing 131 through the gas inlet 133, the gas introduced into the inner space S may be dissolved in the medium filled in the inner space S, and by the gas being dissolved therein, the medium may maintain an appropriate pH level necessary for cell culture.

As a result, since the medium filled in the inner space S can maintain an appropriate pH level required for cell culture, the suspension cells included in the medium can be easily cultured.

Meanwhile, the gas inlet 133 may be covered by a plate-shaped porous member 134 having a predetermined area. That is, the porous member 134 may be attached to the cover 131b to cover the gas inlet 133 formed in the cover 131b.

The porous member 134 may allow passage of gases such as CO₂ while blocking passage of foreign matter and liquids. Accordingly, the medium filled in the inner space S may easily receive a gas necessary for cell culture through the porous member 134 and the gas inlet 133 while other foreign matter is not introduced.

Accordingly, even when a gas is introduced from the outside toward the inner space S through the gas inlet 133, the medium filled in the inner space S may not be contaminated by other foreign matter.

For example, the porous member 134 may be a water-repellent nanofiber membrane. However, the material of the porous member 134 is not limited thereto, and any other material may be used without limitation as long as the material allows passage of gaseous fluids while blocking passage of solid-phase and liquid-phase fluids.

When the suspension cells suspended in the medium come into contact with the plurality of scaffolds 136 and 136′, the plurality of scaffolds 136 and 136′ may transmit signals toward the suspension cells. Accordingly, as described above, the suspension cells included in the medium may repeat a process of being in contact with the scaffolds 136 and 136′ and then detached therefrom while suspended in the medium, and in the process in which the suspension cells are in contact with the scaffolds 136 and 136′ and then detached therefrom, the suspension cells may receive signals from the scaffolds 136 and 136′, receive nutrients from the medium, and grow.

To this end, the scaffolds 136 and 136′ may have surfaces coated with a peptide having a cell proliferation property.

That is, the surfaces of the scaffolds 136 and 136′ may be coated with a peptide motif having the cell proliferation property.

In addition, the scaffolds 136 and 136′ may be provided in a plate shape having a predetermined area to increase an area in which the suspension cells included in the medium may be in contact, and the plurality of scaffolds 136 and 136′ may be disposed in multiple stages and spaced predetermined intervals from each other in the inner space S of the housing 131.

Accordingly, the inner space S may be divided into a plurality of culture spaces S1 by the plurality of scaffolds 136 and 136′ disposed in multiple stages and spaced predetermined intervals in one direction of the housing 131, and the medium including the suspension cells may be stored in the inner space S to fill each of the plurality of culture spaces S1.

In addition, as described above, the medium filled in each of the culture spaces S1 may further include, in addition to the suspension cells, magnetic particles that are peptide motif-coated.

As a result, in the bioreactor 100 for suspension cells according to one embodiment of the present invention, a degree of integration of the scaffolds 136 and 136′ for cell culture may be increased, and since each of the plurality of culture spaces S1 has the plate-shaped scaffolds 136 and 136′ disposed at an upper side and a lower side, an area in which the suspension cells suspended in each of the culture spaces S1 may be in contact with the surfaces of the scaffolds 136 and 136′ may be further increased.

Accordingly, in the bioreactor 100 for suspension cells according to one embodiment of the present invention, a larger amount of cells can be cultured through a single culture process, and because the suspension cells suspended in the culture spaces S1 may be more easily in contact with the surfaces of the scaffolds 136 and 136′, the growth of the suspension cells in the culture spaces S1 divided from one another may be further facilitated. Also, since cell-to-cell interactions and peptide signals may occur more frequently through the magnetic particles included in the medium, the growth of the suspension cells can be promoted.

In addition, in the bioreactor 100 for suspension cells according to one embodiment of the present invention, since the plurality of scaffolds 136 and 136′ may be disposed in multiple stages in a single cell culture part 130, the overall size of the cell culture part 130 at which mass cell culture is possible can be reduced.

Accordingly, since the entire bioreactor 100 for suspension cells according to one embodiment of the present invention can be moved when the frame part 110 having the driving part 120 axially coupled to the cell culture part 130 mounted thereon as described above is moved, mobility of the bioreactor 100 for suspension cells according to one embodiment of the present invention can be secured, and mass cell culture is possible even in small spaces such as a small-scale incubator provided in a laboratory.

In this case, the scaffolds 136 and 136′ may include a nanofiber membrane 136a in which nanofibers are formed in a three-dimensional network structure through electrospinning, and the nanofiber membrane 136a may be provided as a pair of nanofiber membranes 136a to form both surfaces of the scaffolds 136 and 136.

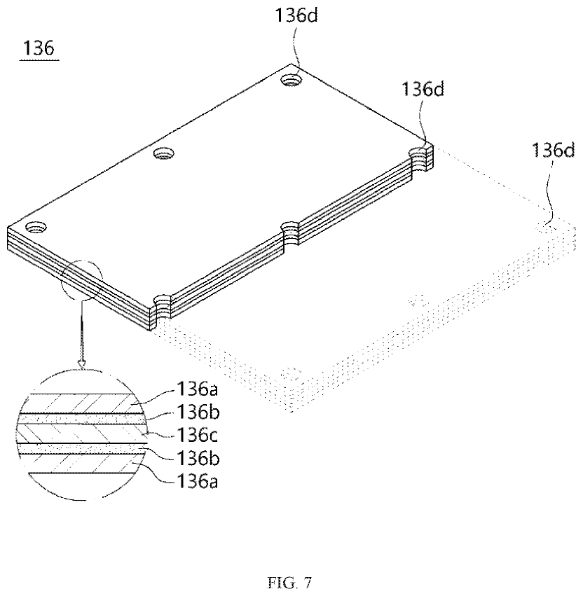

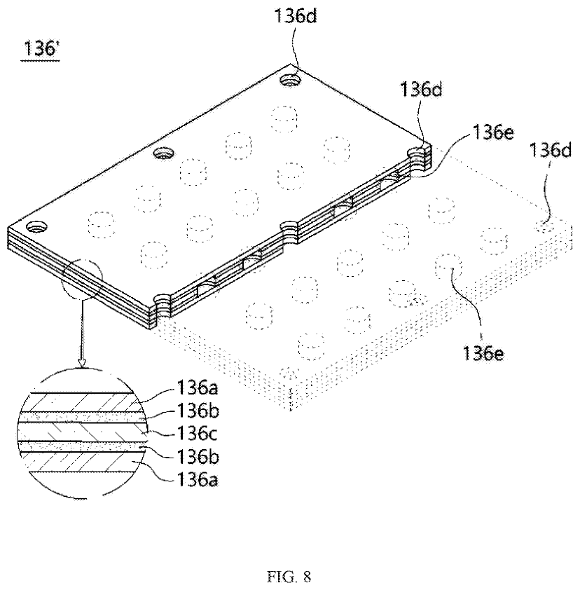

As an example, as illustrated in FIGS. 7 and 8, the scaffolds 136 and 136′ may have a five-layer structure in which the pair of nanofiber membranes 136a are attached to both surfaces of a support member 136c via an adhesive layer 136b.

Here, the support member 136c may be a plate-shaped film member and may support one surface of the nanofiber membrane 136a. Accordingly, the nanofiber membrane 136a may be supported through the support member 136c even when formed in a plate shape having flexibility, and thus bending or drooping of the nanofiber membrane 136a can be prevented. Accordingly, since the scaffolds 136 and 136′ disposed in the inner space S of the housing 131 can maintain an unfolded state, cell culture may be facilitated.

In addition, as described above, the nanofiber membranes 136a forming the surfaces of the scaffolds 136 and 136′ may be plate-shaped nanofiber membranes coated with a peptide motif having a cell proliferation property for the surfaces of the scaffolds 136 and 136′ to have the cell proliferation property.

In this case, in the bioreactor 100 for suspension cells according to one embodiment of the present invention, even when the overall number of the plurality of scaffolds 136 and 136′ arranged in multiple stages to define the plurality of culture spaces S1 in the inner space S increases, a gas necessary for cell culture may be smoothly supplied toward the medium filled in each of the culture spaces S1 defined through the two scaffolds 136 and 136′.

To this end, as illustrated in FIG. 8, the scaffold 136′ may include a plurality of through-holes 136e.

That is, the plurality of through-holes 136e may serve as passages through which the gas introduced from the outside into the inner space S through the gas inlet 133 passes through the scaffold 136′ and moves downward. Accordingly, flowability of the gas may be improved.

To this end, the plurality of through-holes 136e may be formed at portions of the scaffold 136′ that do not allow passage of the gas.

For example, the plurality of through-holes 136e may be formed in the scaffold 136′ to pass through the adhesive layer 136b and the support member 136c in the scaffold 136′.

Accordingly, the gas introduced into the inner space S of the housing 131 through the gas inlet 133 may smoothly move downward through the plurality of through-holes 136e formed in the scaffold 136′.

As a result, the medium filled in each of the culture spaces S1 may smoothly receive a gas through the plurality of through-holes 136e regardless of position.

Meanwhile, in the cell culture part 130, the plurality of scaffolds 136 and 136′ may be integrated into the form of an assembly to smoothly cultivate a large amount of cells by increasing a degree of integration of the plurality of scaffolds 136 and 136′ disposed in the inner space S, to improve assemblability, and to maintain the plurality of culture spaces S1 divided from one another.

For example, the plurality of scaffolds 136 and 136′ may be implemented as a scaffold assembly P, and the scaffold assembly P may have a form in which the plurality of scaffolds 136 and 136′ are integrated while spaced predetermined intervals from each other.

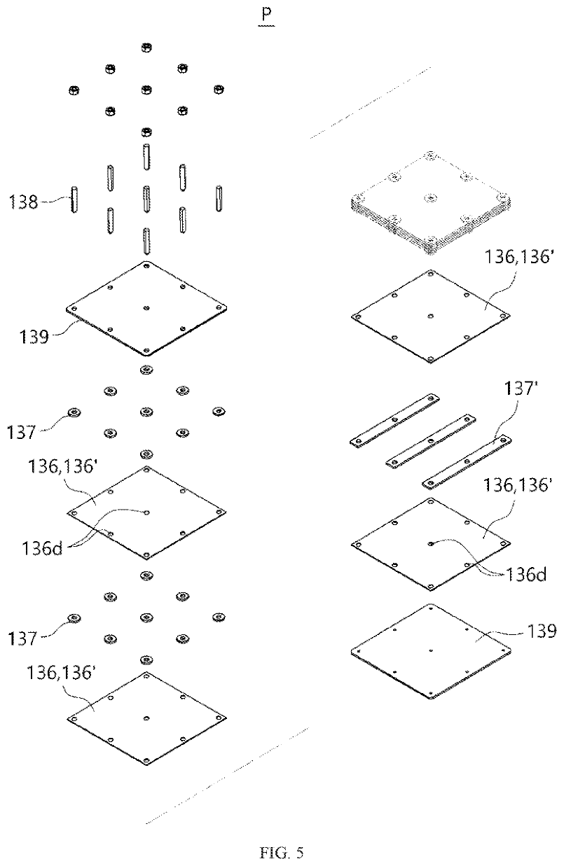

Specifically, as illustrated in FIGS. 4 and 5, the scaffold assembly P may be configured as a stacked type in which the plurality of scaffolds 136 and 136′ are disposed parallel to and spaced from each other in a height direction of the housing 131.

To this end, the scaffold assembly P may include a plurality of fastening bars 138 having a predetermined length and a plurality of spacing members 137 and 137′ fastened to the fastening bars 138, and the plurality of scaffolds 136 and 136′ may be fitted to the fastening bars 138.

Here, as illustrated in FIG. 5, the spacing members 137 and 137′ may be ring-shaped members to be fastened to a single fastening bar 138 or may be bar-shaped members having a plurality of fastening holes formed in a longitudinal direction to be simultaneously fastened to the plurality of fastening bars 138.

In such a case, the plurality of fastening bars 138 may be disposed to be spaced predetermined intervals from each other, and the plurality of fastening bars 138 may have both ends fixed each to support plates 139 having a predetermined area.

Accordingly, the plurality of fastening bars 138 having both ends each fixed to the two support plates 139 may remain spaced predetermined intervals from each other, the plurality of scaffolds 136 and 136′ may be disposed in multiple stages for surfaces thereof to face each other between the two support plates 139, and the plurality of scaffolds 136 and 136′ may be fastened to the fastening bars 138 through a plurality of fastening holes 136d formed at positions corresponding to the plurality of fastening bars 138.

In addition, like the scaffolds 136 and 136′, the plurality of spacing members 137 and 137′ may be fitted to the plurality of fastening bars 138, and the plurality of spacing members 137 and 137′ and the plurality of scaffolds 136 and 136′ may be alternately fastened to the fastening bars 138.

Accordingly, the spacing members 137 and 137′ may be disposed between the two scaffolds 136 and 136′ arranged in the height direction of the housing 131, and by the spacing members 137 and 137′, the two scaffolds 136 and 136′ neighboring each other may remain spaced from each other.

Accordingly, the plurality of culture spaces S1 divided by the two scaffolds 136 and 136′ arranged in the height direction of the housing 131 while having the same height as the spacing members 137 and 137′ may be formed between the plurality of scaffolds 136 and 136′.

However, the scaffold assembly P is not limited thereto, and various other known methods may be applied as long as the plurality of scaffolds 136 and 136′ can remain spaced certain intervals from each other while arranged parallel to each other in one direction.

Meanwhile, in the bioreactor 100 for suspension cells according to one embodiment of the present invention, the rotational speed, rotation cycle, and the like of the driving part 120 may be controlled by a separate controller (not illustrated). Also, the controller may control the overall operation of the bioreactor 100 for suspension cells according to one embodiment of the present invention, in addition to the driving of the driving part 120.

Embodiments of the present invention have been described above, but the spirit of the present invention is not limited by the embodiments presented herein, and those of ordinary skill in the art who understand the spirit of the present invention may easily propose other embodiments by adding other components, changing components, or omitting components within the scope of the same spirit. However, such embodiments also belong to the scope of the spirit of the present invention.

What is claimed is:

1. A bioreactor for suspension cells, the bioreactor comprising:
a frame part;
a driving part mounted on the frame part; and
a cell culture part including a housing having an inner space filled with a medium including suspension cells and a plurality of scaffolds disposed in multiple stages and spaced predetermined intervals from each other in the inner space, the cell culture part having one side axially coupled to the driving part.

2. The bioreactor of claim 1, wherein the cell culture part swings through driving of the driving part for the medium to be moved in a space between two scaffolds neighboring each other.

3. The bioreactor of claim 1, wherein the cell culture part further includes a gas inlet formed to pass through the housing and having a predetermined area to allow a gas to be introduced from the outside into the inner space and a porous member configured to cover the gas inlet to allow the gas to be introduced from the outside into the inner space while preventing the medium filled in the inner space from leaking to the outside.

4. The bioreactor of claim 3, wherein the porous member is a water-repellent membrane.

5. The bioreactor of claim 1, wherein:
the scaffold is a plate-shaped member having a predetermined area;
the inner space is divided into a plurality of culture spaces through the plurality of scaffolds disposed in multiple stages and spaced predetermined intervals in one direction of the housing; and
the medium including the suspension cells is stored in the inner space to fill each of the plurality of culture spaces.

6. The bioreactor of claim 1, wherein:
the scaffold includes a plate-shaped support member having a predetermined area and a pair of nanofiber membranes attached to both surfaces of the support member via an adhesive layer; and
the nanofiber membranes are plate-shaped nanofiber membranes that are motif-coated.

7. The bioreactor of claim 6, wherein the scaffold further includes a plurality of through-holes formed to pass through the support member to facilitate passage of the gas introduced from the outside into the inner space.

8. The bioreactor of claim 1, wherein the plurality of scaffolds remains spaced at predetermined intervals via a spacing member disposed between two scaffolds.

9. The bioreactor of claim 1, wherein the medium further includes magnetic particles that are peptide motif-coated.
